(19) United States
(12) Patent Application Publication
(10) Pub. No.: US 2005/0176778 A1
(43) Pub. Date: Aug. 11, 2005

(54) Isoprenyl derivatives and their use in the title treatment and prevention of osteoporosis and cardiovascular calcification

(75) Inventor: Cees Vermeer, Maastricht (NL)

Correspondence Address: KALOW & SPRINGUT LLP, 488 MADISON AVENUE, 19TH FLOOR, NEW YORK, NY, 10022, US

(73) Assignee: Vitak BV, MD Maastricht (NL)

(21) Appl. No.: 10/770,991

(22) Filed: Feb. 2, 2004

Related U.S. Application Data
(63) Continuation of application No. PCT/EP02/08917, filed on Aug. 5, 2002.

(30) Foreign Application Priority Data
Aug. 3, 2001 (EP) 01202955.9

(51) Int. Cl. C07D 211/84; C07C 50/32; C07C 50/04; A61K 31/12

(52) U.S. Cl. 514/345; 514/682; 514/690; 552/293; 552/294; 552/296; 546/290

(57) ABSTRACT

A non-toxic biologically active compound is provided having the following general formula (I): 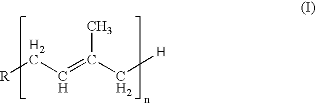
wherein n is an integer from 1 to 14, preferably from 2 to 4, and R is an organic moiety, preferably a group different from but structurally substantially similar to 2-methyl naphthoquinone, or a group P—C(R₁)—P, where each P stands for a —PO(OH)₂ group and R₁ is a (poly)isoprenyl group, hydroxy, halogen (preferably chloro or bromo), or hydrogen, or a pharmaceutically acceptable derivative thereof. These compounds are useful for the treatment or prevention of certain disorders in a mammal, especially a human being, for example postmenopausal loss of bone in women, juvenile or senile osteoporosis in men and women, cardiovascular calcification and other ectopic calcifications.

This is a continuation of International Application PCT/EP02/08917, with an international filing date of Aug. 5, 2002, published in English under PCT Article 21(2), which application is herein incorporated by reference.

### FIELD OF THE INVENTION

The present invention is in the field of organic chemistry, biochemistry, and medicine. More in particular, the invention relates to isoprenyl derivatives and their use to prevent postmenopausal and juvenile or senile loss of bone mass, as well as to prevent calcification of the arteries and heart valves.

### BACKGROUND OF THE INVENTION

Vitamin K is a group name for a number of structurally related compounds which have in common a methylated naphthoquinone group, but which differ in the aliphatic side chain at the 3-position. This may be phytyl (vitamin K-1), geranylgeranyl (menaquinone-4, menatetrenone), or polyisoprenyl (vitamin K-2, menaquinone-n). The classical function of vitamin K is that it serves as a co-factor for γ-glutamyl carboxylase, an endoplasmic enzyme which catalyzes the post-translational carboxylation of glutamate residues into γ-carboxy glutamate (Gla). Gla-residues are calcium binding groups in proteins which are required for the biological activity of the proteins in which they occur.

The active co-factor for γ-glutamyl carboxylase is vitamin K hydroquinone, which is generated by the action of either of two reductases: a dithiol-dependent enzyme which can be completely blocked by coumarin derivatives, and a NADPH-dependent enzyme which is insensitive to coumarins but requires higher intakes of vitamin K. Thus far, the liver is the only tissue in which the NADPH-dependent enzyme system has been demonstrated unequivocally.

During vitamin K-deficiency or treatment with coumarin derivatives, the Gla-residues are not formed, so that the respective proteins are synthesized in an undercarboxylated, i.e. inactive form.

The two major groups of Gla-containing proteins are: (i) certain blood coagulation factors which are synthesized in the liver, and (ii) osteocalcin and Matrix Gla-Protein (“MGP”), two proteins involved in the regulation of tissue calcification which are produced in bone (osteocalcin), cartilage (MGP), and vascular smooth muscle cells (also MGP). The first clinical sign of systemic administration of coumarin derivatives is the (hepatic) synthesis of inactive blood coagulation factors.

To prevent loss of animals due to severe bleeding, a regimen was developed in which animals received a mixture of the coumarin derivative warfarin and vitamin K-1. The latter can be reduced to the active cofactor for y-glutamyl carboxylase by the NADPH-dependent reductase which is present in the liver but could not be demonstrated in bone and vascular tissue.

Animals subjected to this so-called “Vitamin K-1 plus warfarin treatment” developed severe calcifications of the epiphyses [Price, P. et al., Proc. Natl. Acad. Sci. USA 79 (1982) 7734-7738] and other cartilages [Howe, A. M., and Webster, W. S. Teratology 46 (1992) 379-390], decreased growth and osteopathy [Pastoureau, P., et al., J. Bone Miner. Res. 8 (1993) 1417-1426].

A second result was that animals subjected to vitamin K-1 plus warfarin treatment developed substantial calcifications of the arteries and heart valves [Price, P. A., et al., Arterioscler. Thromb. Vasc. Biol. 18 (1998) 1400-1407].

Bisphosphonates are structural analogs of pyrophosphate which competitively inhibit the formation of farnesyl pyrophosphate (farnesyl PP) and geranylgeranyl pyrophosphate (geranylgeranyl PP) in the mevalonate pathway. The resulting farnesyl PP and geranylgeranyl PP are required for prenylation and targeting of essential proteins in signal transduction (e.g. Ras, Rho, Rac). Geranylgeranyl PP is especially used in the activation of osteoclasts, the cells involved in bone resorption [Van Beek E., et al., J. Bone Miner. Res. 14 (1999) 722-729]. Inhibition of prenylation leads to disturbance of signal transduction and apoptosis of osteoclasts, so that postmenopausal bone loss is retarded, and even increase of bone mass is possible.

For these reasons, bisphosphonates are a powerful tool for preventing osteoporosis and related fractures. A drawback is the often reported side-effect of gastrointestinal complaints.

Vascular calcification may occur as a result of atherosclerosis, but also as a result of diabetes mellitus (Mönckeberg's sclerosis) and renal failure. It is the result of two processes: precipitation of calcium salts (often seen in an early stage), and formation of vascular bone tissue. The latter phase is characterized by the presence of osteoblast- and osteoclast-like cells, and a variety of proteins known to occur in bone where they have a function in the control of bone growth and development [Shanahan, C. M., et al., J. Clin. Invest. 93 (1994) 2393-2402, Shanahan, C. M., et al., Circulation 100 (1999) 2168-2176, Boström, K., and Demer, L. L., Crit. Rev. Eukar. Gene Expr. 12 (2000) 151-158]. Whether and how the processes of precipitation and vascular formation of bone tissue are causally related is unknown, but it has been clearly shown that osteoporotic bone loss is associated with a strongly increased risk for vascular calcification [Frye, M. A., et al., Bone and Mineral 19 (1992) 185-194].

It is also known that low estrogen levels in postmenopausal women form a risk factor for both loss of bone mass and accumulation of calcium salts in the vasculature. Hence it seems that calcium metabolism in the bone and vessel wall is regulated by a common principle with opposite effects in both tissues. Further proof for this hypothesis was obtained from the demonstration that bis-phosphonates not only retard osteoporotic bone loss, but also inhibit vascular calcification in the vitamin K1 plus warfarin treatment [Price, P. A., et al., Arterioscler. Thromb. Vasc. Biol. 21 (2001) 817-824].

Several papers disclose that high doses of menaquinone-4 (“MK-4”) almost completely inhibit postmenopausal bone loss [Orimo, H., et al., J. Bone Miner. Metab. 16 (1998) 106-112; Shiraki, M., et al., J. Bone Miner. Res. 15 (2000) 515-521].

The present inventor hypothesized that the geranylgeranyl tail in MK-4 acts as a competitive inhibitor for either the formation of geranylgeranyl PP or one of the further steps in protein prenylation. Therefore, MK-4 may be regarded as a geranylgeranyl derivative in which the pyrophosphorylation is effectively prevented by the presence of the naphthoquinone group. Hence MK-4 will act as an inhibitor of the mevalonate pathway, with a function complementary to that of bisphosphonates.

### SUMMARY OF THE INVENTION

In accordance with one aspect of the present invention a non-toxic biologically active compound is provided having the following general formula (I): 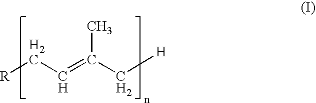
wherein and R is an organic moiety, selected from the group consisting of a naphtho-quinone or benzoquinone derivative (both optionally substituted), with the proviso that R is not a 3-(2-methylnaphthoquinone) moiety; a group P—C(R₁)—P, where each P stands for a —PO(OH)₂ group and R₁ is a (poly)isoprenyl group, hydroxy, halogen (preferably chloro or bromo), or hydrogen; an ester group RmC(O)O—, e.g. a trityl ester, an ether group RPCO—, and a keto group R_{q}C(O)—, where Rₘ, Rₚ an R_{q}, each independently stand for a C₁₋₂₀ alkyl (branched or straight), a C₃₋₁₀ cycloalkyl, a three to ten-membered heterocyclic (having one or more N, O and/or S atoms as hetero atoms), a C₃₋₂₀ cycloalkyl C₁₋₁₀ alkyl, a three to ten-membered heterocyclic C₁₋₁₀ alkyl (having one or more N, O and/or S atoms as hetero atoms), and an aryl or aryl C₁₋₁₀ alkyl, where aryl stands for an aromatic or heteroaromatic (having one or more N, O and/or S atoms as hetero atoms), monocyclic or polycyclic ring system (the latter having preferably two, three or four rings), where all of these groups may be optionally substituted by one or more heteroatoms, such as N, O and/or S, and n is an integer from 1 to 14, preferably from 2 to 4, or a pharmaceutically acceptable derivative thereof. The compounds of formula (I) as defined above are believed to be new, in particular when used as active ingredients in medicaments.

Suitable and preferred groups R include the following structures:

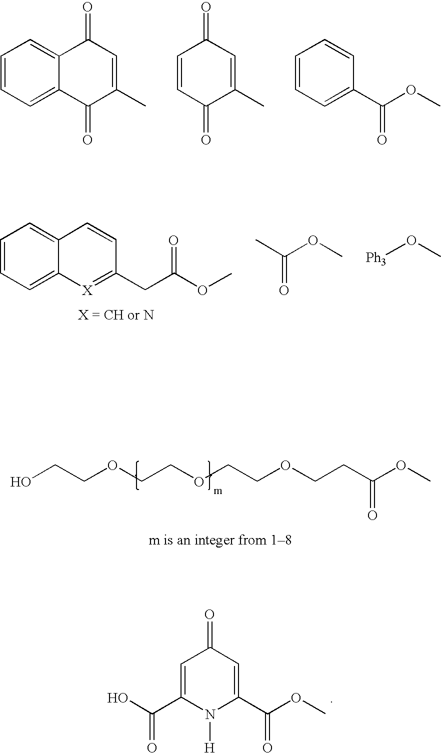

According to another aspect of the invention the use of a compound as defined above, or a pharmaceutically acceptable salt thereof, is provided in the preparation of a medicament for the treatment or prevention of certain disorders in a mammal, especially a human being. Such disorders are selected from the group consisting of postmenopausal loss of bone in women, juvenile or senile osteoporosis in men and women, cardiovascular calcification, including arteriosclerotic and atherosclerotic calcification of the vascular intima, Mönckeberg's sclerosis of the tunica media, and calcification of arterial valves, other ectopic calcifications, such as in pancreatic calcification, renopathy, or malignancies including primary and secondary bone tumors and metastases.

According to a preferred embodiment of the present invention there is provided a combination of at least one compound of formula (I), as defined above, with one or more pharmacologically active substances selected from the group consisting of bisphosphonates, estrogens, calcitonins, and low doses of vitamins D and/or K. In particular the combination of a compound of formula (I) and an N-containing bisphosphonate (e.g., pamdronate, alendronate, olpadronate, thandronate, risedronate, zoledronate, and the like) is believed to be of importance because they inhibit the same reaction or biochemical pathway giving rise to a potential synergistic effect.

According to a further aspect of the invention a pharmaceutical composition is provided comprising, as an active ingredient, a compound of formula (I), as defined above, or a pharmaceutically acceptable salt thereof, in conjunction with a pharmaceutically acceptable carrier.

According to to another aspect of the invention the pharmaceutical composition further comprises one or more pharmacologically active substances selected from the group consisting of bisphosphonates, estrogens, calcitonins, and low doses of vitamins D and/or K, in particular a bisphosphonate compound.

According to still another aspect of the invention a method of treatment is provided for treating or preventing certain disorders in a mammal, especially a human being, which comprises administering to said mammal an effective amount of a compound of formula (I), preferably in conjunction with a pharmaceutically acceptable carrier.

These and other aspects of the present invention will be more fully outlined in the detailed description below.

### DETAILED DESCRIPTION OF THE INVENTION

The present invention is based on the discovery that prenylation of proteins is necessary for signal transduction, and that bisphosphonates inhibit the formation of farnesyl PP and geranylgeranyl PP which is an essential step in protein prenylation.

Prenylation, as used herein, is meant to indicate an addition or substitution reaction by a functionalized isoprenyl or polyisoprenyl moiety. In the case of polyisoprenyl, usually two to four isoprenyl units are present.

Bisphosphonates probably act as structural analogs of the pyrophosphate to be coupled to the polyisoprenyl chains. Notably geranylgeranyl PP is involved in the activation of osteoclasts, resulting in increased bone turnover, and rapid loss of bone mass. On many occasions it has been shown that vascular calcification is regulated by the same cells and the same proteins as those found in bone, but that there is an inverse association between osteoporotic bone loss and vascular calcification. Although the precise mechanism underlying the coupling of both processes has remained unclear until now, they have been shown to be oppositely affected by compounds such as estrogen and bisphosphonates.

Since both estrogen and bisphosphonates have substantial side-effects and are not well accepted or tolerated for long-term treatment, the present inventors have tried to inhibit the formation of geranylgeranyl PP by a related compound having a geranylgeranyl side-chain, 2-methyl 3-geranylgeranyl naphthoquinone, also known as menatetrenone, menaquinone-4, or MK-4. Thus, MK-4 has the structure of vitamin K-1 in which the phytyl side-chain is replaced by geranyl-geranyl (4 isoprenyl residues). This compound was tested in a slightly adapted model for arterial calcification, which is based on blocking the calcification inhibitory activity in the vessel wall and in bone. Since results are visible within one month for the vessel wall, and only after at least nine months for bone, the experiments described here refer to vascular calcification.

MK-4 is the only K-vitamin with a geranylgeranyl side chain, and several studies have demonstrated that the action of high doses of MK-4 (45 mg/day, i.e. 500-1,000× the RDA for vitamin K) on bone is comparable with that of bisphosphonates. Although experimental proof for its mode of action is lacking, the very high dose required and the fact that similar effects were not obtained for vitamin K-1 [Hara, K., et al., Bone 16 (1995) 179-184] make it unlikely that the effects of MK-4 on bone are solely due to its classical vitamin K function.

The compounds according to this invention with formula I as defined above show interesting pharmacological properties, in particular in preventing cardiovascular calcification, which make them potentially useful in medicine to treat or prevent certain disorders, especially in humans.

As will be explained later, the present invention relates in one aspect to compounds lacking vitamin K activity which have retained the potential of interfering with the mevalonate pathway and thus inhibit osteoclast activation. In a preferred group of compounds of formula I the methylated naphthoquinone group of MK-4 is replaced by a group without vitamin K activity. Such groups R (see formula I) include structurally related groups such as (non-methylated) naphthoquinone and benzoquinone, both optionally substituted.

Another preferred group of compounds of formula (I) are new bisphosphonate derivatives having the basic structure P—C(R₁,R₂)—P, where each P stands for a —PO(OH)₂ group and at least one of R₁ and R₂ is a (poly)isoprenyl group as defined in formula (I), and the other one of R₁ and R₂ preferably is the same or a different (poly)isoprenyl group or hydroxy, a halogen (preferably, chloro or bromo), or hydrogen. By linking one or two (poly)isoprenyl groups at the R₁ and/or R₂ position to the central carbon atom of the typical P—C(R₁,R₂)—P structure of bisphosphonates, a new generation of more powerful bisphosphonates is provided which is effective at much lower doses thereby preventing or reducing gastrointestinal problems. Preferred compounds which are expected to be active as competitive inhibitors of geranylgeranyl PP formation are those containing the polyisoprenoid backbone of either geranyl or farnesyl or geranylgeranyl, which are shown in the following structural formulas:

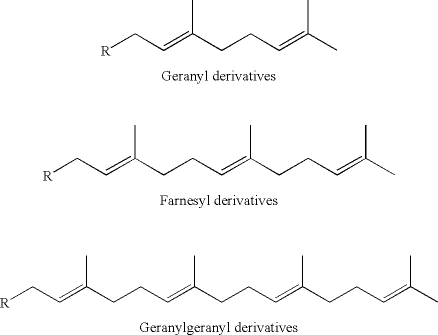

Biologically active compounds used for pyrophosphate formation are preferably alcohols. Therefore, active inhibitors according to the present invention preferably have, in formula (I), a group R at the comparable OH-position, which is either directly coupled to the first carbon atom of the polyisoprenyl moiety or which includes, for instance, an ether or ester linkage.

Since MK-4 counteracts oral anticoagulation, preferred compounds according to the present invention are for example geranyl, farnesyl, and geranyl-geranyl derivatives with a similar structure, such as non-methylated 3-geranyl-geranyl naphthoquinone, geranylgeranyl benzoquinone, and trityl-geranylgeraniol.

The compounds of formula (I) as defined above can be prepared in various ways. The methods as such are known in the art for the preparation of structurally related or similar compounds, for example vitamin K2. Some of these methods have been reviewed in EP-A-0243849, the disclosure of which is incorporated herein by reference. According to one approach a polyprenyl alcohol compound, such as geranyl alcohol or farnesyl alcohol which are commercially available, is coupled to a compound of the formula RH or a functibnalized derivative thereof, preferably in the presence of a suitable catalyst, e.g. a an acid Friedel-Crafts catalyst. Appropriate measures should be taken to protect double bonds or other groups which may interfere with the coupling reaction. This is all known to a person skilled in the art who will be able to perform such reactions without inventive skill or undue experimentation. The same applies to other methods known in the art, such as the coupling of the “R” moiety to isoprene epoxide, to convert the allylic alcohol so obtained to a bromide and then extending the polyisoprenyl chain by reaction with, e.g., the corresponding p-tolylsulfone derivative.

Also, the method disclosed in EP-A-0243849 can be used which comprises stepwise building up a polyprenoid moiety while the same is attached to a terminal group, or building up such polyprenoid moiety and coupling it to a desired terminal group.

Compounds of formula (I) which have an ester function can also be prepared by esterification where the (poly)isoprenyl moiety is usually provided as a commercially available) alcohol, and the “R” part which sometimes is a mimic of 2-methylnaphtoquinone is usually provided as an acid, such as a derivative of benzoic acid, 1-naphthylacetic acid, quinaldic acid, chelidamic acid, and the like.

The compounds of formula (I) can be present, with reference to the (first) double-bond, viewed from the R group, in the isoprenyl chain, in Z form (also called cis-form) or in E-form (also called trans form) or as a mixture of these two forms. Usually the Z-form is biologically less active, if not even inactive, and therefore the E-form generally is preferred.

The compounds of formula (I) may comprise one or more asymmetric carbon atoms, giving rise to enantiomeric forms, or mixture(s) thereof. It should be understood that all such enantiomeric and/or E- or Z-forms, whether in a substantially single form or a mixture, in purified or non-purified form, are encompassed by the present invention.

Processes for preparing vitamins of the vitamin K1 and K2 series in their E-isomeric form also belong to the state of the art; see, e.g., EP-A-0015436 and U.S. Pat. No. 4,320,065, U.S. Pat. No. 4,374,290 and U.S. Pat. No. 4,374,775. The methods disclosed in these references may also be used for the preparation of the E-form of compounds of formula I, with some modifications which are evident to a skilled person.

A pharmaceutical composition comprising a compound of formula I and/or one or more pharmaceutically acceptable derivatives thereof as an active ingredient is suitably administered to humans by way of oral or parenteral administration. Pharmaceutically acceptable derivatives, as used herein, are meant to include any form of the active substance of formula (I) as defined above, which is suitable for administration to a mammal, in particular a human. Such pharmaceutically acceptable derivatives include the usual acid addition salts, but also ester and amide forms, and the like, where appropriate. It should be realized that a compound of formula (I) or a pharmaceutically acceptable derivative thereof may also comprise solvates of such compounds, such as, e.g., a hydrate. All such forms are known to a person skilled in the art, who can also make an appropriate selection without inventive skill.

The medicament can be administered in conventional form for oral administration, e.g. as tablets, lozenges, dragees and capsules. However, for the administration of the drug to children, should the occasion arise, it may be preferred to formulate the composition as an oral liquid preparation such as a syrup, a nasal spray, or a suppository. The medicament can also be administered parenterally, e.g. by intramuscular or subcutaneous injection, using formulations in which the medicament is employed in a saline or other pharmaceutically acceptable, injectable composition.

An amount effective to treat the disorder hereinbefore described depends on the usual factors such as the nature and severity of the disorder being treated, the weight of the patient, the specific compound(s) of choice, and considerations and preferences of the prescriber. The amount of active ingredient(s) to be administered usually will be in the range of micrograms up to 100 mg or more per dose. However, a unit dose will normally contain 1 to 1000 mg, suitably 1 to 500 mg, for example an amount in the range of from 2 to 400 mg such as 2, 5, 10, 20, 30, 40, 50, 100, 200, 300 and 400 mg of the active ingredient. Unit doses will normally be administered once or more than once per day, for example 1, 2, 3, 4, 5 or 6 times a day, more usually 1 to 4 times a day, such that the total daily dose is normally in the range, for a 70 kg adult, of 1 to 1000 mg, for example 1 to 500 mg, that is in the range of approximately 0.01 to 15 mg/kg/day, more usually 0.1 to 6 mg/kg/day, for example 1 to 6 mg/kg/day.

It is greatly preferred that the compound of formula I and/or pharmaceutically acceptable derivative(s) thereof according to the invention is administered in the form of a unit-dose composition, such as a unit dose oral, sub-lingual, rectal, topical or parenteral (especially intravenous) composition.

Such compositions are prepared by admixture and are suitably adapted for oral or parenteral administration, and as such may be in the form of tablets, capsules, oral liquid preparations, powders, granules, lozenges, reconstitutable powders, injectable and infusable solutions or suspensions or suppositories. Orally administrable compositions are preferred, in particular shaped oral compositions, since they are more convenient for general use. The preparation of such compositions is well known to people skilled in the art and can be optimized in a routine way without exerting inventive skill and without undue experimentation.

Tablets and capsules for oral administration are usually presented in a unit dose, and contain conventional excipients such as binding agents, fillers, diluents, tabletting agents, lubricants, disintegrants, colorants, flavourings, and wetting agents. The tablets may be coated according to well known methods in the art.

Suitable fillers for use include cellulose, mannitol, lactose and other similar agents. Suitable disintegrants include starch, polyvinylpyrrolidone and starch derivatives such as sodium starch glycollate. Suitable lubricants include, for example, magnesium stearate. Suitable pharmaceutically acceptable wetting agents include sodium lauryl sulphate.

These solid oral compositions may be prepared by conventional methods of blending, filling, tabletting or the like. Repeated blending operations may be used to distribute the active agent throughout those compositions employing large quantities of fillers. Such operations are, of course, conventional in the art.

Oral liquid preparations may be in the form of, for example, aqueous or oily suspensions, solutions, emulsions, syrups, or elixirs, or may be presented as a dry product for reconstitution with water or other suitable vehicle before use. Such liquid preparations may contain conventional additives such as suspending agents, for example sorbitol, syrup, methyl cellulose, gelatin, hydroxyethylcellulose, carboxymethyl cellulose, aluminium stearate gel or hydrogenated edible fats, emulsifying agents, for example lecithin, sorbitan monooleate, or acacia; non-aqueous-vehicles (which may include edible oils), for example, almond oil, fractionated coconut oil, oily esters such as esters of glycerine, propylene glycol, or ethyl alcohol; preservatives, for example methyl or propyl p-hydroxybenzoate or sorbic acid, and if desired conventional flavouring or colouring agents.

Oral formulations further include controlled release formulations which may also be useful in the practice of this invention. The controlled release formulation may be designed to give an initial high dose of the active material and then a steady dose over an extended period of time, or a slow build up to the desired dose rate, or variations of these procedures. Controlled release formulations also include conventional sustained release formulations, for example tablets or granules having an enteric coating.

Nasal spray compositions are also a useful way of administering the pharmaceutical preparations of this invention to patients such as children for whom compliance is difficult. Such formulations are generally aqueous and are packaged in a nasal spray applicator which delivers a fine spray of the composition to the nasal passages.

Suppositories are also a traditionally good way of administering drugs to children and can be used for the purposes of this invention. Typical bases for formulating suppositories include water-soluble diluents such as polyalkylene glycols and fats, e.g. cocoa oil and polyglycol ester or mixtures of such materials.

For parenteral administration, fluid unit dose forms are prepared containing the compound and a sterile vehicle. The compound, depending on the vehicle and the concentration, can be either suspended or dissolved. Parenteral solutions are normally prepared by dissolving the compound in a vehicle and filter sterilising before filling into a suitable vial or ampoule and sealing. Advantageously, adjuvants such as a local anaesthetic, preservatives and buffering agents are also dissolved in the vehicle. To enhance the stability, the composition can be frozen after filling into the vial and the water removed under vacuum.

Parenteral suspensions are prepared in substantially the same manner except that the compound is suspended in the vehicle instead of being dissolved and sterilised usually by exposure to ethylene oxide before suspending in the sterile vehicle. Advantageously, a surfactant or wetting agent is included in the composition to facilitate uniform distribution of the compound of the invention.

As is common practice, the compositions will usually be accompanied by written or printed directions for use in the medical treatment concerned.

In the present invention the “Vitamin K1 plus warfarin” model was used to test effects of isoprenyl derivatives on vascular calcification. This model is also referred to as the “KW model for calcification” or “KW model”. Because of the parallels between vascular calcification and calcium metabolism in bone, the model is regarded to be valid for describing potential effects on bone. Whereas in this model the bone deformations will not become visible before 9 months of treatment, calcification of the large arteries is usually apparent within 4 weeks of treatment. This model therefore provides an excellent in vivo model to test the compounds according to the invention for their tissue distribution, toxicity and effectivity. The effectiveness of this approach was demonstrated by using relatively small amounts of MK-4 in combination with an excess of vitamin K-1. It was shown that calcification was not inhibited by vitamin K-1 in doses going up from 40 to 600 mg/kg of body weight per day. The highest dose was 15,000 times the amount required for normal blood coagulation in untreated animals, and 15 times the amount required for normal blood coagulation in warfarin-treated animals. At these high intakes all tissues contain excessive amounts of vitamin K-1, which apparently are incapable of supporting the synthesis of inhibitors of calcium precipitation such as the Gla-protein MGP. In the same model the addition of 10 mg/kg body weight per day of MK-4 effectively inhibited all vascular calcification, i.e. it had a similar effect as were reported for bisphosphonates.

It is to be noted that since MK-4 possesses vitamin K activity, it will be involved in the production of Gla-proteins, some of which may act as calcification inhibitors. The observed effects under the conditions chosen (excess of vitamin K-1) suggest, however, that MK-4 has a second activity which is not related to its naphthoquinone group, but which is completely different from the classical function of vitamin K. (The present invention is inter alia based on the discovery of this second activity). Hence the treatment of subjects and patients with MK-4 in order to prevent or to cure osteoporosis and/or vascular calcification seems to be indicated. A drawback of such treatment is that patients at risk for thrombosis often receive vitamin K-antagonists to counteract severe cardiovascular events such as myocardial infarction and stroke. Treatment of such patients with MK-4 or any other substance showing vitamin K activity may therefore result in a high mortality in this patient group. As stated before, the present invention relates to compounds and compositions lacking vitamin K activity which have retained the potential of interfering with the mevalonate pathway and thus inhibit osteoclast activation.

The following experimental work further illustrates the present invention, but should not be regarded as limiting its scope in any respect.

### BRIEF DESCRIPTION OF THE EXPERIMENTS

In experiment 1 the dietary requirement of vitamin K-1 was measured in untreated male Lewis rats, 6 weeks of age, on the basis of blood coagulation tests after a treatment period of 4 weeks. At an intake of 40 microgram vitamin K-1 per kg body weight per day, the synthesis of coagulation factors had reached its (maximal) plateau level.

In experiment 2 the dietary requirement of vitamin K-1 in warfarin-treated (300 mg Vitamin K-1 per kg body weight per day) male Lewis rats, 6 weeks of age, was measured on the basis of blood coagulation tests after a treatment period of 4 weeks. At a level of 40 mg vitamin K-1 per kg body weight per day a normal prothrombin synthesis was maintained.

In experiment 3 vascular calcification (as demonstrated by Von Kossa staining) in rats treated with warfarin (300 mg/kg/d) and increasing doses of vitamin K-1 was analyzed. After 4 weeks of treatment, substantial calcification was found at vitamin K-1 doses of 20, 40, 70, 100, 300, and 600 mg vitamin K-1 per kg body weight per day, with no visible improvement in the higher dose groups.

In experiment 4 vascular calcification (as demonstrated by Von Kossa staining) in rats treated with warfarin (300 mg/kg/d) and vitamin K-1 (70 mg/kg/d) and increasing doses of MK-4 was analyzed. After 6 weeks of treatment severe calcification was observed in the controls (no MK-4), but no calcification was found in animals treated with 10, 20, and 50 mg of MK-4 per kg body weight per day.

Experiment 1

Male rats of the Lewis strain were used throughout our experiments, six animals per group. Thirty six animals entered the experiment at the age of 6 weeks and received a vitamin K deficient diet (Hope Farms, Woerden, The Netherlands) for one week to deplete them from endogenous vitamin K. During the experiment they were housed in coprophagy-preventing cages. After this period citrated blood was taken from the tail vein, and the prothrombin concentrations (one-stage coagulation assay) were checked to be below the normal reference value. At that time they received vitamin K-deficient food to which controlled amounts of vitamin K-1 were added in the following doses: 0.1, 0.2, 0.4, 0.6, 0.8, and 1.0 mg/kg of food. The daily food intake was recorded, and the body weight of the animals was measured regularly. After four weeks of treatment at the indicated doses again blood was taken and prothrombin concentrations were measured. Mean prothrombin concentrations in the groups were: 23%, 48%, 82%, 98%, 102%, and 99% of pooled normal rat reference plasma. Based on these data it was decided that the diet containing 0.6 mg of vitamin K-1 per kg supplied sufficient vitamin K to maintain normal prothrombin synthesis. This amount corresponds with an intake of 40 micrograms of vitamin K-1 per kg body weight per day.

Experiment 2

Thirty six male Lewis rats (6 weeks of age) were treated with an excess of warfarin (300 mg vitamin K-1 per kg body weight per day) to block all dithiol reductase present in the tissues. They were subdivided into groups of 6 animals each and received vitamin K-1 in the following doses: 20, 40, 70, 100, 300, and 600 mg/kg body weight per day. Plasma prothrombin levels were measured at this time: at 20 and 40 mg of vitamin K-1 per day the circulating prothrombin concentration was 60% of normal, at all higher intakes the values were comparable to normal reference values. It was concluded that 70 mg of vitamin K-1 per kg body weight per day effectively protects the animals against warfarin-induced bleeding.

Experiment 3

The same animals described in experiment 2 were killed, and the aortic arch and carotid arteries were removed and stained for calcium deposits using the Von Kossa staining and histochemical inspection. Calcification of the media (notably the elastic lamelae) was found in all animals, showing that even at very high concentrations-vitamin K-1 was unable to counteract calcification, whereas it had effectively counteracted impairment of blood coagulation factor synthesis.

Experiment 4

Twenty four animals received the calcifying regimen of warfarin (300 mg/kg/d) and vitamin K-1 (70 mg/kg/d), but in addition MK-4 in increasing doses: 0, 10, 20, and 50 mg per kg body weight per day. After 6 weeks of treatment the animals were killed, the aortae and carotid arteries were removed and used for histochemical analysis after Von Kossa staining for calcification. Whereas in the control group (no MK-4) severe calcification was observed, no calcification was found in animals treated with 10, 20, and 50 mg of MK-4 per kg body weight per day.

1. A non-toxic biologically active compound having the following general formula (I): 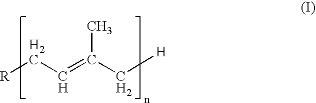
wherein an R is an organic moiety, selected from the group consisting of:
a substituted or unsubstituted naphthoquinone or benzoquinone derivative;
a group P—C(R₁)—P, where each P is a —PO(OH)₂ group and R₁ is a (poly)isoprenyl group, a hydroxy, a halogen, or a hydrogen;
an ester group RₘC(O)O—;
an ether group RₚCO—; and
a keto group R_{q}C(O)—;
where Rₘ, Rₚ and R_{q}, are each independently a straight or branched C₁₋₂₀ alkyl; a C₃₋₁₀ cycloalkyl; a three to ten-membered heterocyclic group having one or more N, O, and/or S atoms as hetero atoms; a C₃₋₂₀ cycloalkyl C₁₋₁₀ alkyl; a three to ten-membered heterocyclic C₁₋₁₀ alkyl having one or more N, O and/or S atoms as hetero atoms; or an aryl or aryl C₁₋₁₀ alkyl, where aryl represents an aromatic or heteroaromatic, having one or more N, O and/or S atoms as hetero atoms, monocyclic or polycyclic ring system, where all of these ring systems can comprise one or more heteroatoms; and
wherein n is an integer from 1 to 14, or a pharmaceutically acceptable derivative thereof.

2. A compound according to claim 1, wherein R has one of the following structures:

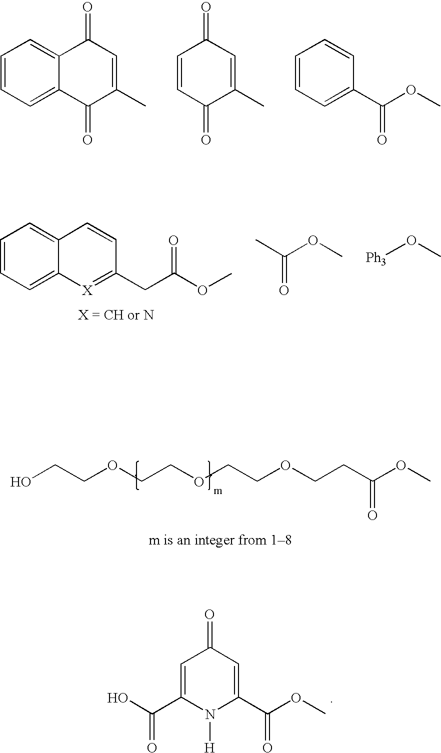

3. The compound according to claim 1, wherein at least one of the naphthoquinone derivative and the benzoquinone derivative is substituted.

4. The compound according to claim 1, wherein the halogen is chlorine or bromine.

5. The compound according to claim 1, wherein the ester group RₘC(O)O— is a trityl ester.

6. The compound according to claim 1, wherein the polycyclic ring system comprises two, three, or four rings.

7. The compound according to claim 1, wherein one or more of the ring systems comprise at least one heteroatom that is an O, an S, or an N.

8. The compound according to claim 1, wherein n is an integer from 2 to 4.

9. A pharmaceutical composition comprising the compound of claim 1 and a pharmaceutically acceptable carrier.

10. The pharmaceutical composition according to claim 9, further comprising one or more pharmacologically active substances selected from the group consisting of bisphosphonates, estrogens, calcitonins, and low doses of vitamins D and/or K.

11. The pharmacuetical composition according to claim 10, wherein the pharmacologically active substance is a low dose of vitamin K.

12. A method of treating a disorder in a mammal using the compound of claim 1 or a pharmaceutical composition comprising said compound.

13. The method according to claim 12, wherein the mammal is a non-human mammal.

14. The method according to claim 12, wherein the mammal is a human.

15. The method according to claim 12, wherein the disorder is postmenopausal loss of bone in women, juvenile or senile osteoporosis in men and women, cardiovascular calcification, ectopic calcification, renopathy, malignancies, primary bone tumors, secondary bone tumors, or metastases.

16. The method according to claim 15, wherein the cardiovascular calcification is arteriosclerotic calcification, atherosclerotic calcification of the vascular intima, Möbnckeberg's sclerosis of the tunica media, or calcification of arterial valves.

17. The method according to claim 15, wherein the ectopic calcification is pancreatic calcification.

18. The method according to claim 12, wherein the pharmaceutical composition is administered in a pharmaceutically effective amount.
